(19) United States
(12) Patent Application Publication
(10) Pub. No.: US 2021/0113317 A1
(43) Pub. Date: Apr. 22, 2021

(54) VETERINARY ANESTHESIA INDUCTION NOSE CONE ASSEMBLY

(71) Applicant: William L. Connelly, Kingman, AZ (US)

(72) Inventor: William L. Connelly, Kingman, AZ (US)

(21) Appl. No.: 16/915,833

(22) Filed: Jun. 29, 2020

Related U.S. Application Data
(60) Provisional application No. 62/916,678, filed on Oct. 17, 2019.

(51) Int. Cl. A61D 7/04; A61M 16/01; A61M 16/06; A61M 16/20

(52) U.S. Cl. CPC A61D 7/04; A61M 16/01; A61M 2250/00; A61M 16/208; A61M 16/0683

(57) ABSTRACT

A veterinary anesthesia induction cone assembly may include a chamber housing comprising a rim at a first end for receiving a snout of an animal, and a second end opposite the first end. A diaphragm may be releasably and mateably coupled with the rim of the chamber housing to form a first diaphragm opening. A second end of the diaphragm may be opposite the lip, be disposed within the chamber housing, and be configured to allow a nose of the animal to receive gas flow from the chamber housing. A diaphragm sidewall may connect, and extend between, the first diaphragm opening and the second diaphragm opening. A retaining strap may be coupled to the chamber housing and positionable between a first position for fitting the retaining strap around a head of an animal and a second position for storing the retaining strap.

### CROSS REFERENCE TO RELATED APPLICATIONS

This application claims the benefit, including the filing date, of U.S. provisional patent application No. 62/916,678 to William L. Connelly titled “Veterinary Anesthesia Induction Nose Cone Assembly” filed Oct. 17, 2019, the entirety of the disclosure of which is incorporated herein by this reference.

### TECHNICAL FIELD

This application relates to the field of anesthesia apparatus and more particularly to veterinary anesthesia apparatus.

### BACKGROUND

For decades, conventional induction cones have just included a simple cover over the entire muzzle end of the cone. The cover has a single slit in the middle thereof to receive a muzzle of an animal in an attempt to provide a seal between the cone and the muzzle. Additionally, conventional induction cones are not provided with a way to attach the cone to an animal. For example, if professionals need to administer oxygen to an animal, they have to improvise a head strap of some sort out of gauze or other materials on-hand.

### SUMMARY

According to aspects of the disclosure, a veterinary anesthesia induction cone assembly may comprise a chamber housing comprising a rim at a first end for receiving a snout of an animal and a second end opposite the first end. A diaphragm may comprise a compliant lip configured to be releasably and mateably coupled with the rim of the first end of the chamber housing to form a first diaphragm opening. A second end of the diaphragm may be opposite the lip and disposed within the chamber housing. The second end of the diaphragm may comprise a second diaphragm opening opposite the first diaphragm opening and be configured to allow a nose of the animal to receive gas flow from the chamber housing. The second diaphragm opening may be smaller than the first diaphragm opening. A diaphragm sidewall may connect with, and extend between, the first diaphragm opening and the second diaphragm opening. A retaining strap may be moveably coupled to the chamber housing and positionable between a first position for fitting the retaining strap around a head of an animal wearing the veterinary anesthesia induction cone and a second position for storing the retaining strap around the chamber housing.

Particular embodiments may comprise ends of the retaining strap further being attached to, and directly contacting, the chamber housing. The strap may further comprise a center pad disposed between the first end of the strap and the second end of the strap and a storage attachment device adjacent the center pad. The diaphragm may comprise a telescoping diaphragm with a length less than a length of the chamber housing. The chamber housing may comprise a tapered form that facilitates the stacking or nesting of multiple chamber housings with each other for compact storage, shipping, or transport. The chamber housing may comprise a frustoconical shape. The second diaphragm opening may be moveable between a first position adjacent the first diaphragm opening and a second expanded position away from the first diaphragm opening.

According to another aspect of the disclosure, a veterinary anesthesia induction cone assembly may comprise a chamber housing comprising a first end for receiving a snout of an animal and a second end opposite the first end. A diaphragm may comprise a first end configured to be releasably and mateably coupled with the first end of the chamber housing to form a first diaphragm opening. The diaphragm may comprise a second end opposite the first end and disposed within the chamber housing. The second end of the diaphragm may comprise a second diaphragm opening opposite the first diaphragm opening, and be configured to allow a nose of the animal to receive gas flow from the chamber housing. A diaphragm sidewall may connect and extend between, the first diaphragm opening and the second diaphragm opening.

Particular embodiments may further comprise a retaining strap moveably coupled to the chamber housing and positionable between a first position for fitting the retaining strap around a head of an animal wearing the veterinary anesthesia induction cone and a second position for storing the retaining strap around the chamber housing. The chamber housing may comprise a groove formed around a circumference of the chamber housing, and the retaining strap may comprise a ring of material mateably coupled with the groove formed around the circumference of the chamber housing. A 1-way port or valve may be disposed within the chamber housing and be configured to allow for exhalation of the animal to go outside the chamber housing. The chamber housing may comprise a tapered form that facilitates the stacking or nesting of multiple chamber housings with each other for compact storage, shipping, or transport. The second diaphragm opening may be moveable between a first position adjacent the first diaphragm opening and a second expanded position away from the first diaphragm opening.

According to yet another aspect of the disclosure, a veterinary anesthesia induction cone assembly may comprise a chamber housing comprising a first end for receiving a snout of an animal and a second end opposite the first end. A diaphragm may be coupled to, and disposed within, the chamber housing. The diaphragm may comprise a diaphragm opening disposed within the chamber housing and be configured to allow a nose of the animal to receive gas flow from the chamber housing. A retaining strap may be moveably coupled to the chamber housing and be positionable between a first position for fitting the retaining strap around a head of an animal wearing the veterinary anesthesia induction cone and a second position for storing the retaining strap around the chamber housing.

Particular embodiments may further comprise a diaphragm sidewall extending from the second diaphragm opening and configured to envelope the snout of the animal within the chamber housing. The chamber housing may comprise a groove formed around a circumference of the chamber housing, and the retaining strap may comprise a ring of material mateably coupled with the groove formed around the circumference of the chamber housing. Ends of the retaining strap may be attached to, and directly contact, the chamber housing. The strap may further comprise a storage attachment device disposed between the first end of the strap and the second end of the strap. The chamber housing may comprise a tapered form that facilitates the stacking or nesting of multiple chamber housings with each other for compact storage, shipping, or transport. The diaphragm opening may be moveable between a first position adjacent the first end of the chamber housing and a second expanded position away from the first end of the chamber housing.

The foregoing and other aspects, features, applications, and advantages will be apparent to those of ordinary skill in the art from the specification, drawings, and the claims. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographer, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of 35 U. S.C. § 112(f). Moreover, even if the provisions of 35 U.S.C. § 112(f) are invoked to define the claimed aspects, it is intended that these aspects not be limited only to the specific structure, material or acts that are described in the embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent to those of ordinary skill in the art from the specification, drawings, and the claims.

### BRIEF DESCRIPTION OF THE DRAWINGS

Implementations will hereinafter be described in conjunction with the appended drawings, where like designations denote like elements, and:

FIGS. 1A-1C illustrate various views of a veterinary anesthesia induction cone assembly;

FIGS. 2A-2G illustrate various views of a diaphragm or gasket coupled to a chamber housing or cone of the veterinary anesthesia induction cone assembly of claims 1A-1C;

FIGS. 3A-3D illustrates various views of a strap of the veterinary anesthesia induction cone assembly of claims 1A-1C;

FIG. 4 illustrates a veterinary anesthesia induction cone assembly in use on an animal;

FIGS. 5A and 5B illustrate ports or valves for use with a veterinary anesthesia induction cone assembly;

FIGS. 6A and 6B illustrate various views of another aspect of a veterinary anesthesia induction cone assembly;

FIG. 7A-7J illustrates various views of another aspect of a veterinary anesthesia induction cone assembly.

### DETAILED DESCRIPTION

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

While this disclosure includes a number of implementations in many different forms, there is shown in the drawings and will herein be described in detail particular implementations with the understanding that the present disclosure is to be considered as an exemplification of the principles of the disclosed methods and systems, and is not intended to limit the broad aspect of the disclosed concepts to the implementations illustrated.

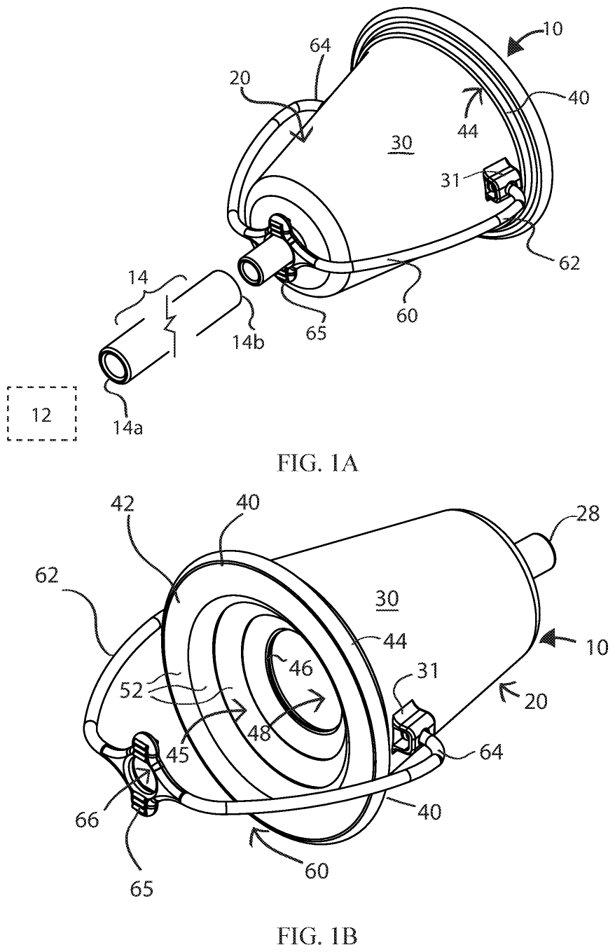

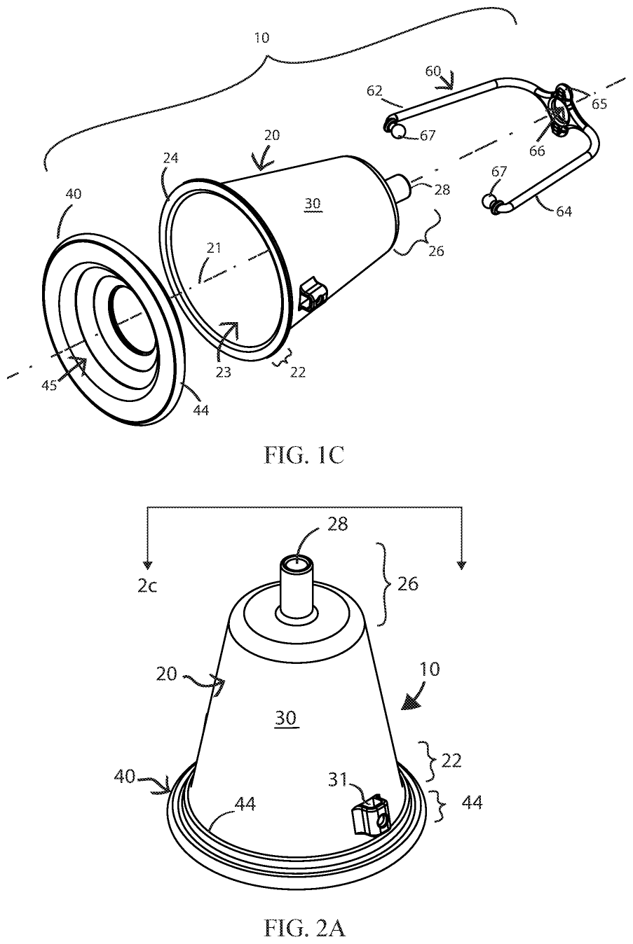

FIGS. 1A-1C illustrate various views of a veterinary anesthesia induction cone assembly 10, which for convenience may also be referred to as an anesthesia cone assembly or cone assembly. FIG. 1A shows the cone assembly 10 may comprise a chamber housing 20, which may for convenience also be referred to as a housing, chamber, or cone. The chamber housing 20 may comprise a rim 24 at a first end 23 for receiving a snout of an animal and a second end 26 opposite the first end 23.

The term “cone” as used herein with respect to the cone assembly 10 is not limited to a geometric cone, and as such includes any suitable 3-dimensional (3D) volume for receiving an animal nose, snout, muzzle, beak, proboscis, or member (referred to collectively hereinafter as “nose” or “snout”). The “cone” may include any 3D form that tapers from a larger radius or cross-sectional area at the base to a smaller radius or cross-section area at a distal end, such as, or including, a frustoconical shape. A tapered shape of the chamber housing 20 may provide a number of benefits, including generally following a contour of the nose of the animal inserted into the assembly 10, as well as facilitating stacking or nesting of numerous chamber housings 20 one on top of another, such as during storage, shipping, or transport.

The chamber housing 10 may be formed of glass, plexiglass, or other translucent or transparent material; plastics and/or other like materials; fiberglass or other fiber based material; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; elastomers and/or other like materials; rubbers (synthetic and/or natural); metals, and/or other like materials; composites and/or other like materials, and/or any combination or composite of the foregoing.

The chamber housing 10 may be made by, with, or involve 3-D printing, extrusion, pultrusion, vacuum forming, injection molding, blow molding, resin transfer molding, casting, forging, cold rolling, milling, drilling, reaming, turning, grinding, stamping, cutting, carving, bending, welding, soldering, hardening, riveting, punching, plating, and/or the like.

The second end or distal end 26 of the chamber housing may be coupled to a tube or line 14 for supplying a fluid or gas to the animal receiving anesthesia. The fluid supplied to the animal may be oxygen, a sedative or anesthesia, a combination of the above, or any other suitable gas which may be supplied from a tank, supply, or gas supply 12. As such, the tube 14 may comprise a first end 14a for coupling to the gas supply 12, such as a tank or line, and a second end 14b of tube 14 for coupling to the veterinary anesthesia induction cone 10 or chamber housing 20, such as at a second opening or supply tube port or member 28. In some instances, the supply tube port 28 may be disposed at, and extend through, the second end 26 of the chamber housing 20, and be in-line with the first opening 23 in the chamber housing 20, as well as being in-line with the nose of the animal using the cone assembly 10. In other instances, the supply tube port 28 may not be in-line with the first opening 23 in the chamber housing 20, as well as not being in-line with the nose of the animal using the cone assembly 10. Instead, the supply tube port 28 may be offset or formed in a chamber sidewall or sidewall 30 of the chamber housing 20. The chamber sidewall 30 may connect, and extend between, the first end 22 of the chamber housing 20 and the second end 26 of the chamber housing 20.

The anesthesia induction cone assembly 10 may further comprise a diaphragm, gasket, or cover 40 coupled to, and disposed or nested at least partially within, the chamber housing 20. The diaphragm 40 may be formed of compliant, deformable, or elastic material that can be overmolded on at least a portion of the chamber housing 20. The diaphragm 40 may be formed of, or comprise, one or more materials, such as plastics and/or other like materials; thermosets (such as epoxy, phenolic resin, polyimide, polyurethane, and/or the like), and/or other like materials; elastomers and/or other like materials; rubbers (synthetic and/or natural) and/or other like materials; fiberglass or other fiber based material, including carbon fiber, Kevlar, or other similar materials; polymers such as thermoplastics (such as ABS, fluoropolymers, polyacetal, polyamide, polycarbonate, polyethylene, polysulfone, and/or the like; foams as well as composites and/or other like materials, and/or any combination or composite of the foregoing.

In some instances, conventional rubber with its distinctive smell, which may be bothersome to animals, can be avoided or neutralized with a neutral or pleasant smell, or other material used for the benefit of the animal using the cone assembly 10. Additionally, the diaphragm 40 may also be formed or infused with an aromatic material or scent that is pleasant or calming to the animal-patient, such as to replace a naturally occurring rubber or plastic-like smell that may be bothersome or disruptive to the patient. In some instances, oils will be infused with the material of the diaphragm 40, such as essential oils or aromatic oils, which will include without limitation Lavender Oil or other suitable oil. In some instances, a compartment, pouch, chamber, or filter may be included within, or as part of, the cone assembly 10, so as to provide a changeable aromatic for the user. Designing for control of smells for the user may also be true of any other components of the cone assembly 10.

The diaphragm 40 may be made by, with, or involve 3-D printing, extrusion, pultrusion, vacuum forming, injection molding, blow molding, resin transfer molding, casting, cutting, and/or the like.

The diaphragm 40 may comprise a compliant lip 44 configured to be releasably and mateably coupled with the rim 24 of the first end 22 of the chamber housing 20 to form a first diaphragm opening 45. The diaphragm 40 may also comprise a second end 46 opposite the lip 44, the second end 46 being disposed or nested within the chamber housing 20, and offset from the first opening 23 of the chamber housing 20. The second end 46 may further comprise a second diaphragm opening 48 opposite the first diaphragm opening 45, the second diaphragm opening 48 configured to allow a nose of the animal to receive gas flow from the gas supply 12 and through the chamber housing 20. The second diaphragm opening 48 may be smaller than the first diaphragm opening 45, and a diaphragm sidewall 50 may connect, and extending between, the first diaphragm opening 45 and the second diaphragm opening 48. The second diaphragm opening 48, or an area of the second diaphragm opening 48 may be smaller than the first diaphragm opening 45 or an area of the first diaphragm opening.

By forming the diaphragm or gasket 40 with a compliant, deformable, cushioning, or soft material, the diaphragm 40 may cushion a skull of the animal against the chamber sidewall or cone 30, which can be hard, and thus may decrease a chance of causing one or more of irritation, bruising, and nerve damage to the animal.

The anesthesia induction cone assembly 10 may further comprise strap or retaining strap 60 for comfortably, firmly, and releasably coupling the cone assembly 10 to the head of the animal using the cone assembly 10. The retaining strap 60 may be coupled to, or directly contact, the chamber housing 20, the diaphragm 30, or both.

The retaining strap 60 may be formed of one or more compliant, deformable, or elastic materials. The retaining strap 60 may be formed of, or comprise, one or more materials, such as plastics and/or other like materials; thermosets (such as epoxy, phenolic resin, polyimide, polyurethane, and/or the like), and/or other like materials; elastomers and/or other like materials; rubbers (synthetic and/or natural) and/or other like materials; fiberglass or other fiber based material, including carbon fiber, Kevlar, textiles, elastic cords, cloth, string, webbing, cordage, or other similar synthetic or natural materials; polymers such as thermoplastics (such as ABS, fluoropolymers, polyacetal, polyamide, polycarbonate, polyethylene, polysulfone, and/or the like; foams as well as composites and/or other like materials, and/or any combination or composite of the foregoing.

The retaining strap 60 may be made by, with, or involve 3-D printing, extrusion, pultrusion, vacuum forming, injection molding, blow molding, resin transfer molding, casting, turning, stamping, cutting, carving, bending, weaving, sewing, welding, soldering, punching, plating, and/or the like.

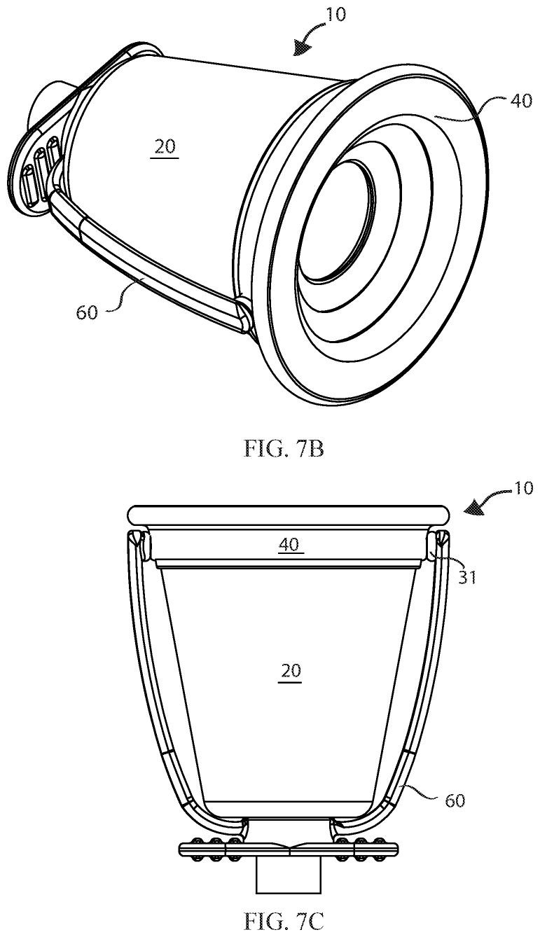

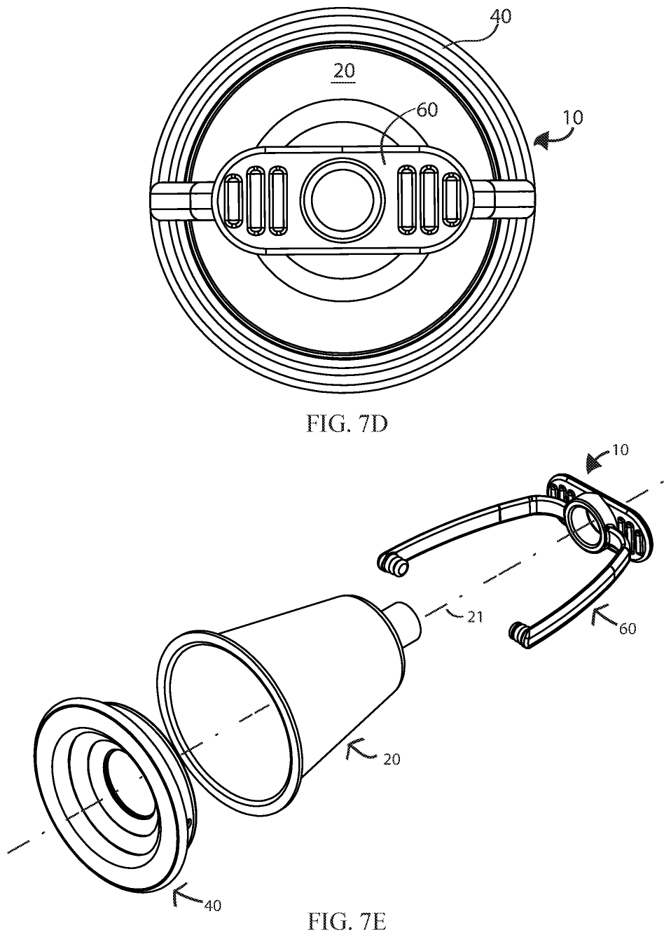

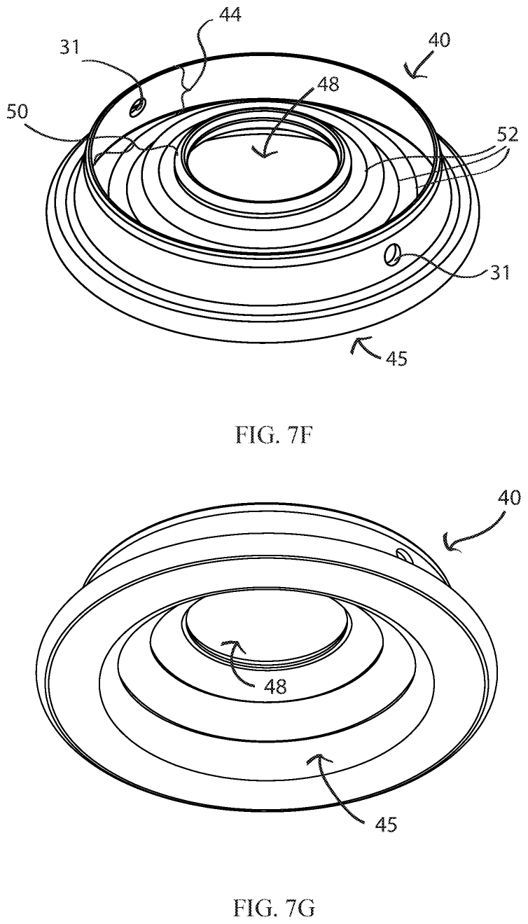

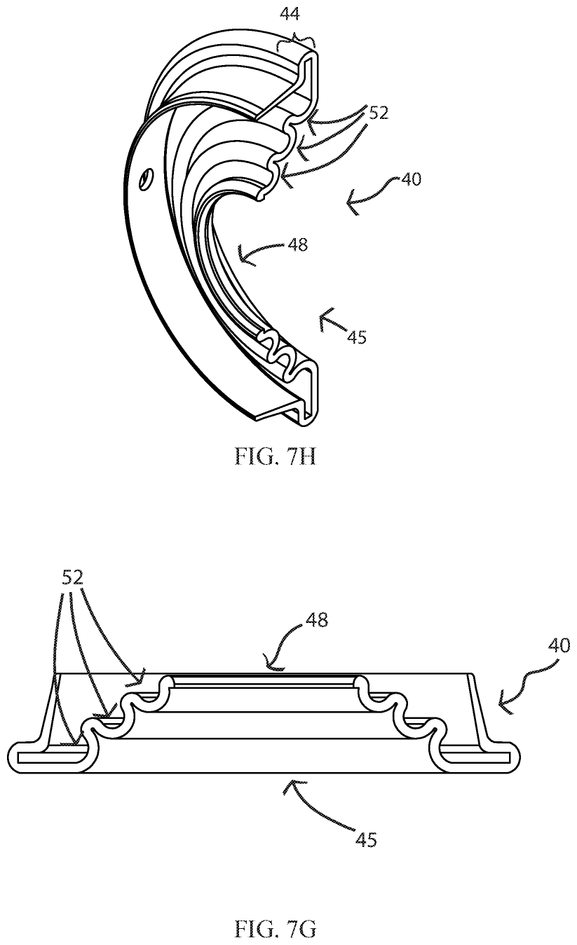

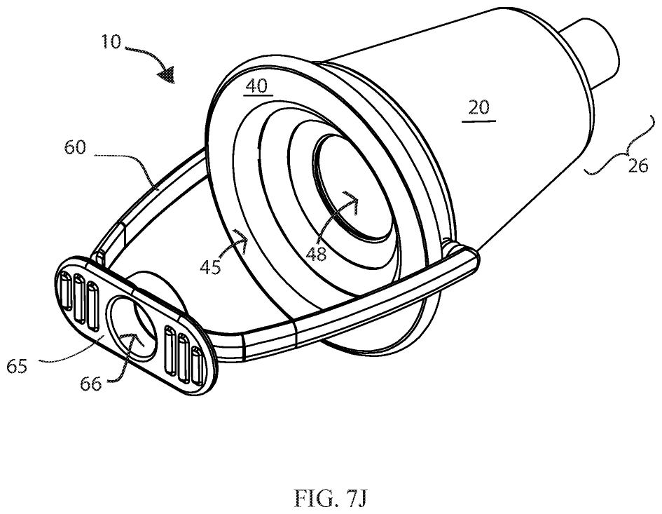

The retaining strap 60 may be moveably coupled to the chamber housing 20 and positionable between a first position (as shown, e.g., in FIG. 1B) for fitting the retaining strap 60 around a head of an animal wearing the veterinary anesthesia induction cone 10, and a second position (as shown, e.g., in FIG. 1A) for storing the retaining strap 60 around the chamber housing 20. The ends 62, 64 of the retaining strap 60 may be attached to, and directly contact, the chamber housing 20, as shown in FIGS. 1A and 1B. In other instances, the ends 62, 64 of the retaining strap 60 may be attached to, and directly contact, the diaphragm 40 as shown, e.g., in FIGS. 7A-7J.

The retaining strap 60 may comprise a first end 62 and a second end 64, with a center pad 65 disposed between the first end 62 and the second end 64. The center pad 65 may be wider than the strap, thereby distributing force at a lower pressure across the head of the user to make wearing of the cone assembly 10 more comfortable. The center pad 65 may comprise an opening 66 through the center pad 65, the opening 66 being sized to fit, or mateably coupled with, the second end 26 of the chamber housing 20 or the second opening or supply tube port or member 28. As such, the opening or other suitable or temporary attachment device 66 may allow the strap 60 to be coupled to the chamber housing 20 when not in use, and to keep the strap 60 conveniently out of the way and tangle free while stored and readily accessible for subsequent use, as is shown in FIG. 1A. The strap 60 may further comprise one or more strap attachment devices 67 for coupling the strap 60 to the housing 20 or diaphragm 40. In some instances, the strap 60 may comprise two strap attachment devices 67, such as one at the first end 62 of the strap 60 the other at the second end 64 of the strap 60.

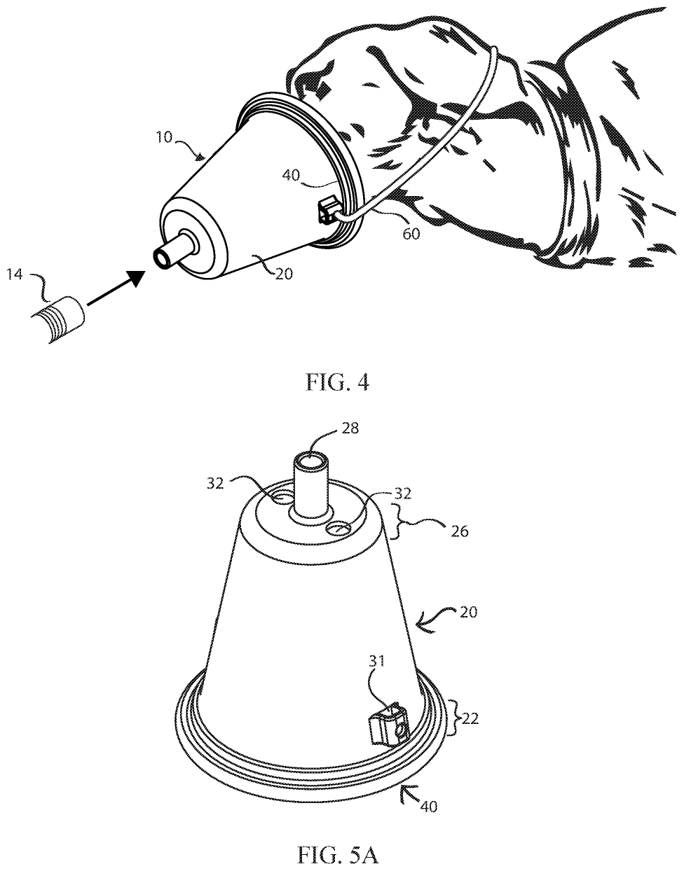

FIG. 1B, shows a perspective view of the cone assembly 10, similar to the view shown in FIG. 1A, but flipped so that the first end 22 of the chamber housing 20 and the diaphragm 40 is shown on the left side of the figure rather than on the right side as in FIG. 1A. As can be seen, e.g., in FIGS. 1B and FIG. 4, when the nose of the animal user is placed through the first diaphragm opening 45 and extends into the diaphragm 40, along the diaphragm sidewall 50, and towards the second diaphragm opening 48, the diaphragm or gasket 40 may form a customized and comfortable seal for the wearer that also prevents leakage of gas from the cone apparatus 10, directing the gas supplied from the gas supply 12 to the nose of the user while improving uptake and reducing waste. In other words, the diaphragm sidewall 50 can extend from, or between, the first diaphragm opening 45 and the second diaphragm opening 48 and be configured to envelope the snout of the animal within the chamber housing 20.

The diaphragm or gasket 40 may be telescoping or extendable/retractable and comprise a number of steps or bellows 52 that may be formed of any desirable shape or cross-sectional shape, including circular, angular, square, rectangle, square, oval, or any other geometric or organic shape according to a desirable configuration or design. The shapes of the bellows 52 may refer to the shapes between the first end 42 and the lip 44 that attaches to the first end 22 of the chamber housing 20 and the second diaphragm opening 48 for receiving the nose and allowing passage of the gas to the nose. As such, the bellows 52 may be formed as a series of concentric, nested, or telescoping shapes. In some instances, portions of the bellows 52 or of the diaphragm sidewall 50 will be made of more deformable and less deformable material to form rigid areas and jointed areas between adjacent rigid areas that allow the bellows to extend, expand, and retract, or contract, whether radially, axially, or both (such as in a direction along a centerline 21of the cone 20 or in a direction between the first end 22 and the second end 26 of the chamber hosing 20).

As such, in instances where a nose of the animal is longer, the bellows 52 of the diaphragm or gasket 40 may extend a greater distance, and when the nose is shorter, the bellows 52 of the diaphragm or gasket 40 may extend a shorter distance. Stated another way, the second diaphragm opening 48 is moveable between a first position adjacent the first diaphragm opening 45 and a second expanded position away from the first diaphragm opening 45. In any event, the diaphragm 40 comprises a length Ld less than a length of the chamber housing Lh.

The nose cone assembly 10 may also be provided in multiple sizes, to accommodate different sized pets or animals on which the assembly will be used. In some instances, two or three sizes of the cone assembly 10 may be available for different sized animals, although a person of ordinary skill in the art (POSA) will appreciate that any number of sizes may be made available. For example, a large, medium, and small sized cone assemblies 10 may be available, as well as any size therebetween or thereabout and even larger or smaller sizes. The large size of the cone assembly 10 may comprise a chamber housing comprising a maximum chamber housing diameter Dh of, or about, 7.5 inches, the medium size comprising a maximum chamber housing diameter Dh of, or about, 5.5 inches, and the small size comprising a maximum chamber housing diameter Dh of, or about, 2.5 inches. As used herein, “about,” “thereabout,” or approximately means within a percent difference in a range of 0-40%, 0-30%, 0-20%, 0-10%, or 0-5% of the stated value.

In some embodiments, the diaphragm 40 may be over molded onto the chamber housing 20, or a portion thereof (like rim 24), so as to permanently, non-removably, or non-temporarily be affixed to the chamber housing 20, such that the gasket need not ever be removed from the cone. In other instances, the diaphragm 40 may be non-permanently, removably, releasably, or temporarily affixed to the chamber housing 20. The diaphragm 40 and chamber housing 20 may also be releasably or permanently coupled to the chamber housing 20 with suitable mechanical fasteners such as screws or other suitable devices, rather than merely being friction fit or press fit together.

As noted above, FIG. 1B differs from FIG. 1A in that the retaining strap 60, while coupled to the diaphragm 40, is rotated away from the cone so as to be positioned for being disposed over a head or neck of an animal wearing the cone so as to hold the cone over the animal's nose. The transition of the strap 60 from a storage position to an active or worn position may be facilitated by strap attachment device 67 positioned at or near both the first end 62 and the second end 64 of end of the strap 60, which as shown in FIGS. 1A and 1B, may be coupled with the chamber housing 20.

A length of the strap 60 may also be adjusted by a slide, pull, cinch, belt, or by other suitable arraignment, such as by elastic deformation of the strap itself, in order to accommodate different sizes of pets and pet heads to which the cone assembly 10 will be applied. In addition to the tightening or loosening of the strap 60, the bellows or movement of the diaphragm 40 can also accommodate different sizes of heads and noses and a comfortable and customizable fit to the animal or individual wearing the nose cone assembly 10. Thus, the rotating or adjustable strap provides a convenient way for a person, individual, or entity to apply the cone assembly 10 (and administer a fluid or gas, such as oxygen) to the user, without needing to use tape, gauze, string, straps, line, or other material to customizable attach of the nose cone assembly 10 on the user's nose.

In accordance with the above, a method of using the induction nose cone assembly 10 may comprise: providing a cone assembly 10, such as by removing the cone assembly 10 from a stack of other nose cone assemblies; moving or rotating the strap 60 from a stowed position (as shown in FIG. 1A) to an deployed position (as shown in FIG. 1B); placing the first end 22 of the chamber housing 20 and the diaphragm 40 of the nose cone assembly 10 over a nose of an animal so that the diaphragm sidewall 50 extends along the animal's nose to or into the second diaphragm opening 48; placing the deployed strap around the head or neck of the animal; adjusting a length, position, or both, of the strap to the head of the animal; attaching a fluid or gas supply line 14 to the gas supply 12 and the nose cone assembly 10; and initialing a flow of fluid or gas to the animal through the supply line 14 and to the animals' nose within the cone assembly 10.

FIG. 1C illustrates an exploded view of the anesthesia induction cone assembly 10 with the chamber housing 20, the diaphragm 40, and the strap 60 spread out along the centerline 21 of the chamber housing 20.

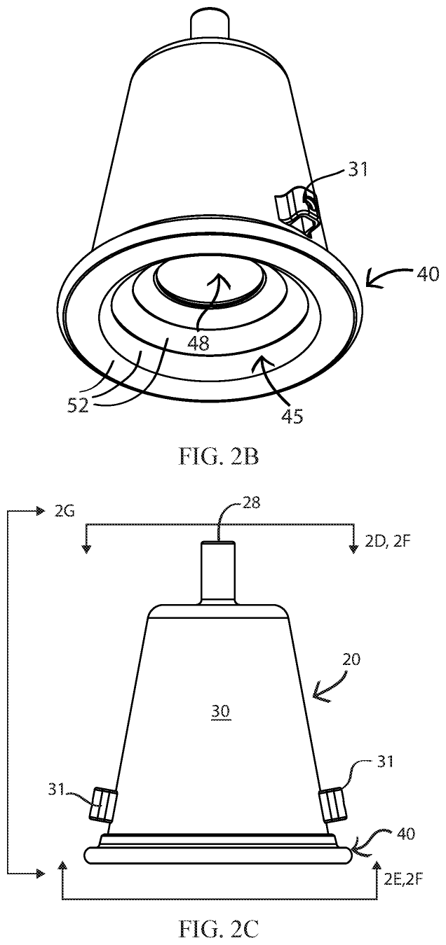

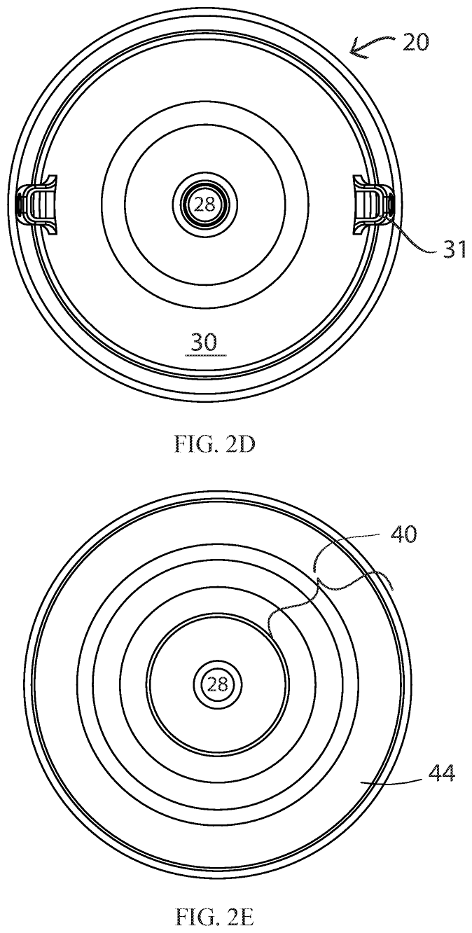

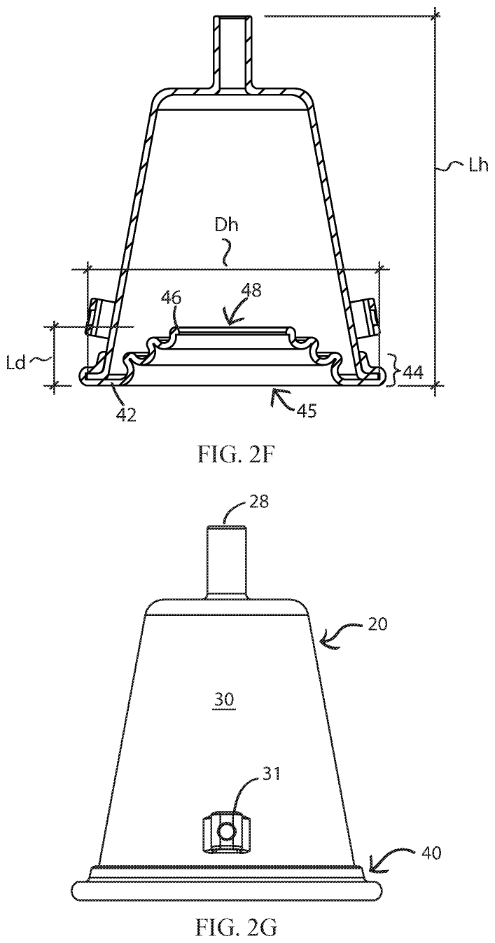

FIGS. 2A-2G illustrate various views of the anesthesia induction cone assembly 10 without the strap 60, or in other words the diaphragm 40 coupled to the chamber housing 20, similar to what was shown in FIGS. 1A-1C. FIG. 2A illustrates a side perspective view of the anesthesia induction cone assembly 10 in which the diaphragm 40 is shown on the bottom of the figure and the first diaphragm opening 45 is oriented away from the viewer.

FIG. 2B illustrates another side perspective view of the anesthesia induction cone assembly 10 in which the diaphragm 40 is shown on the bottom of the figure and the first diaphragm opening 45 is oriented towards the viewer. Between the first or larger diaphragm opening 45 and the second or smaller diaphragm opening 48, a number of steps or bellows 52 are shown formed along the diaphragm sidewall 50.

FIG. 2C illustrates a side profile view of the anesthesia induction cone assembly 10, as taken along the view line 2C in FIG. 2A. In FIG. 2C, the diaphragm 40 is shown at the bottom of the figure. Strap attachment structures 31 are shown on, in, or coupled to, opposing sides of an outer surface of the chamber housing 20, and configured to receive, or to be mateably coupled with, the strap attachment devices 67.

FIG. 2D illustrates a top plan view of the anesthesia induction cone assembly 10, as taken along the view line 2D in FIG. 2C.

FIG. 2E illustrates a bottom plan view of the anesthesia induction cone assembly 10, as taken along the view line 2E in FIG. 2C.

FIG. 2F illustrates a cross-sectional side view of the anesthesia induction cone assembly 10, as taken along the section lines 2F in FIG. 2C. FIG. 2F further includes illustrative dimensions for the length Ld of diaphragm 40, the length Lh of the chamber housing 20, and the maximum chamber housing diameter Dh.

FIG. 2G illustrates a side view of the anesthesia induction cone assembly 10, as taken along the view line 2G shown in FIG. 2C, which is a view similar to the view shown in FIG. 2C, but rotated 90 degrees from the view shown in FIG. 2C. FIG. 2G shows the strap attachment structure 31 disposed on, in, or coupled to, the front of the outer surface of the chamber housing 20, and configured to receive, or to be mateably coupled with, the strap attachment devices 67.

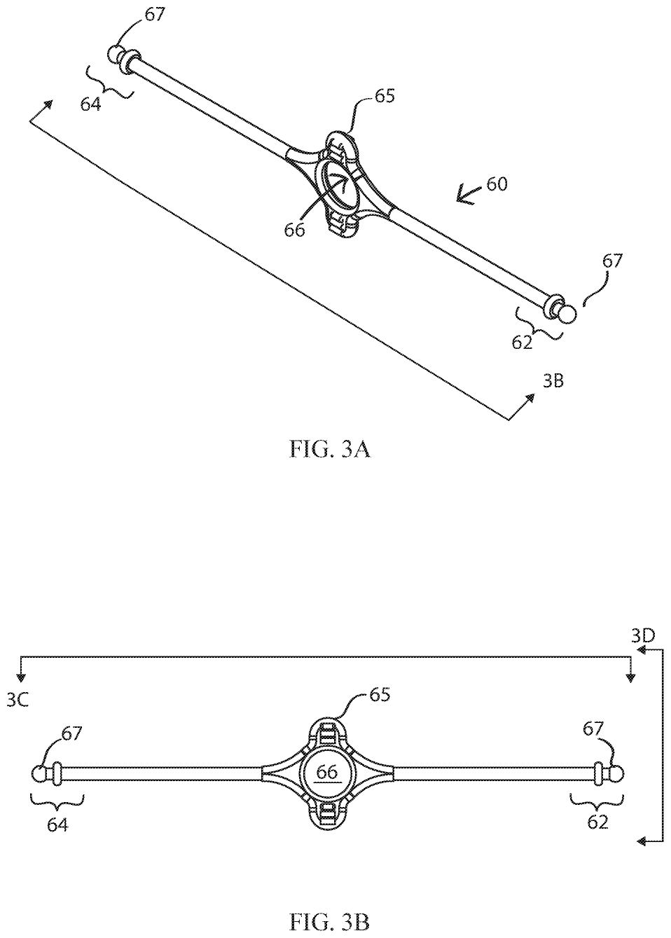

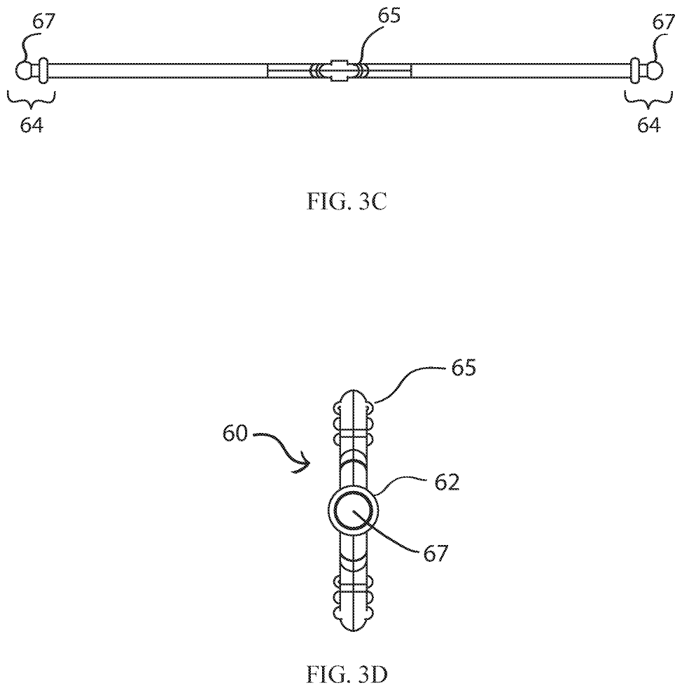

FIGS. 3A-3D illustrates various views of the strap 60 of the veterinary anesthesia induction cone assembly 10 that was shown in FIGS. 1A-1C, and may couple to the chamber housing 20 or the diaphragm 40 for coupling the cone assembly 10 around of head or neck or an animal to hold the cone assembly 10 in place during use.

FIG. 3A shows a perspective view of the strap 60 with a coupling member or opening 66 formed in the center of the pad 65 of the strap 66. The coupling member 66 may comprise a circular shape or any suitable shape such as oval, square, rectangle, or any other geometric or organic form, that may mateably couple with the second, small opening or supply tube port or member 28 of the chamber housing 20. As shown, strap material, including pad 65, may be formed completely around the coupling member 66, while in other instances a break or opening may extend through the strap material to opening or coupling member 66. In yet other instances, a snap, clasp, button, hook, hook and loop fasteners, or other mechanical fastener may be used to temporarily or releasable couple the strap 60 to the chamber housing 20 or the diaphragm 40. The coupling member 66 may be centered at a center or central portion of the strap 60 as shown, and in other instances may also be offset or not centered with respect to the strap 60 or a length of the strap 60.

FIG. 3A also shows ends 62, 64 of the strap 60 may comprise attachment features or devices 67 for coupling the ends of the strap 60 to the diaphragm 40, or to a portion of the chamber housing 20. The attachment features 67 may comprise one or more of a pin, screw, ridge, snap, clasp, button, hook, or other mechanical fastener that may provide suitable mechanical attachment while being able to move between a stowed and deployed position, such as by being able to: 1) rotate and be installed/stowed over the second end 26 of the chamber housing 20 for when the nose cone assembly is not in use (e.g. for storage); and 2) be pulled off the stowed end, rotated, and then stretched over an animal's head when the nose cone assembly 10 is in use (e.g. if you need to administer oxygen or anesthesia to an animal).

FIG. 3B shows another view of the strap 60 separated from the diaphragm 40 and the chamber housing 20. More specifically, FIG. 3B shows a side profile view of the strap 60 taken along the view line 3B shown in FIG. 3A.

FIG. 3C shows another view of the strap 60 separated from the diaphragm 40 and the chamber housing 20. More specifically, FIG. 3C shows a top plan view of the strap 60 taken along the view line 3C shown in FIG. 3B.

FIG. 3D shows another view of the strap 60 separated from the diaphragm 40 and the chamber housing 20. More specifically, FIG. 3D shows a side or end view of the strap 60 taken along the view line 3F shown in FIG. 3B.

FIG. 4 illustrates an example of the veterinary anesthesia induction cone assembly 10 in use on a dog, with the nose of the dog positioned within the chamber housing 20, the nose near or extending beyond the second diaphragm opening 48. The supply line 14 is shown coupled to the chamber housing 20 for providing a gas supply to the chamber housing 20 and the dog. The strap 60 extends around the head of the dog to secure the cone assembly 10 to the dog. The diaphragm 40 provides a good comfortable seal that is airtight or nearly airtight with the snout of the dog, and prevents undesired leakage of the gas from between the nose of dog and the cone assembly 10.

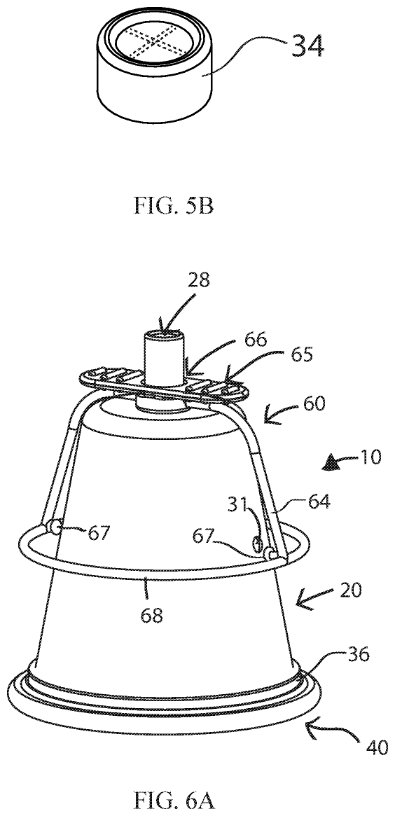

FIGS. 5A and 5B illustrate valve ports or valves 32 with valve inserts 34 for use with the veterinary anesthesia induction cone assembly 10. FIG. 5A shows the valves 32 may comprise 1-way port or valves disposed within the chamber housing 20, such as at the second end 26 of the chamber housing 20, and configured to allow for exhalation by the animal to outside of the chamber housing, so as to vent CO₂. The valve port 32 may comprise a valve insert 34, which may be a 1-way port or valve, venturi structure, or insert that is disposed within, or through, an opening through the chamber housing 20, and is exposed to both the space inside of, and outside of, the chamber housing 20.

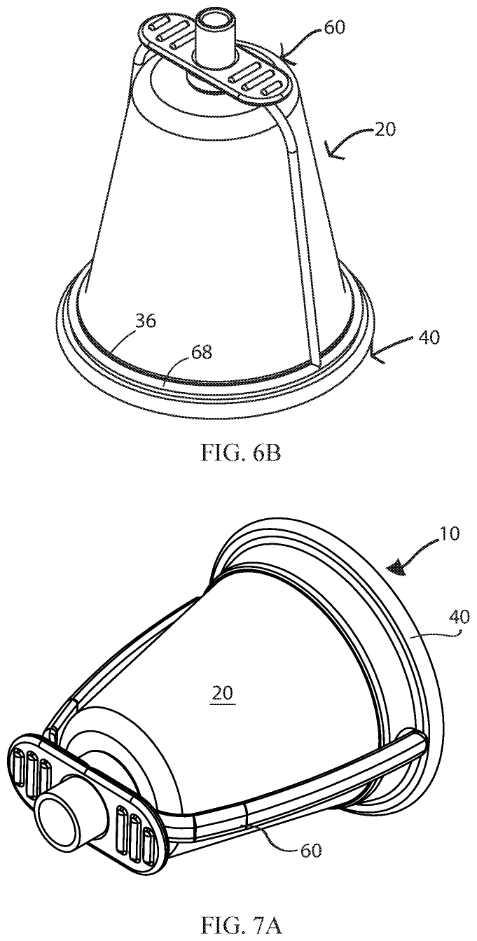

FIGS. 6A and 6B illustrate side perspective views of the cone assembly 10 illustrating another aspect of coupling the strap 60 to the of a veterinary anesthesia induction cone assembly 10. More specifically, FIGS. 6A and 6B show the retaining strap 60 comprising the strap attachment device 67 formed as a ring of material 68 sized and configured to be disposed around an outer diameter of the chamber housing 20. FIG. 6A shows the ring 68 may comprise, or be coupled to, rivets or other suitable attachment device that couples to the corresponding openings/other suitable attachment devices 31 on, in, or within the chamber housing 20, the diaphragm 40, or both. FIG. 6B shows the chamber housing 20 may comprise a groove or channel 36 formed around a circumference of the chamber housing 20, and that the retaining strap 60 may comprise a ring 68 of material mateably coupled with the groove or channel 36 formed around the circumference of the chamber housing 20. In either event, the strap 60 may move, pivot, or rotate from a storage position to an in-use position.

FIG. 7A-7J illustrates various views of another aspect of a veterinary anesthesia cone assembly 10, in which the strap attachment device 67 directly contacts the diaphragm 40 (or strap attachment structures 31 formed thereon) rather than the chamber housing 20 (or strap attachment structures 31 formed thereon), as shown in FIGS. 1A-2G. FIG. 7A illustrates a side perspective view in which the diaphragm 40 is shown on the right side of the figure with the first diaphragm opening 45 disposed away from the viewer. FIG. 7A also shows the retaining strap 60 in a storage position with the ends of the strap coupled to the improved diaphragm 40 near or at the base of the chamber housing 20, the strap 60 wrapped around the chamber housing 20 with the coupling member or opening 66 in the strap 60 coupled to the second end 26 of the chamber housing 20.

FIG. 7B illustrates another a side perspective view (similar to the view of FIG. 7A) in which the diaphragm 40 is shown on the right side of the figure, but differs from FIG. 7A by the first diaphragm opening 45 being disposed towards the viewer. FIG. 7A also shows the retaining strap 60 in a storage position.

FIG. 7C illustrates a side profile view of the cone assembly 10 shown in FIGS. 7A and 7B.

FIG. 7D illustrates a top plan view of the cone assembly 10 shown in FIGS. 7A-7C.

FIG. 7E illustrates an exploded view of the anesthesia induction cone assembly 10 shown in FIGS. 7A-7D, but with the chamber housing 20, the diaphragm 40, and the strap 60 spread out along the centerline 21 of the chamber housing 20.

FIG. 7F illustrates a perspective view of the diaphragm 40 without the chamber housing 20 and the strap 60. In FIG. 7F, the diaphragm 40 is shown with the first or large diaphragm opening 45 at the bottom of the figure, and the smaller second diaphragm opening 48 disposed at the top of the figure.

FIG. 7G illustrates a perspective view of the diaphragm 40 (without the chamber housing 20 and the strap 60) with the diaphragm 40 flipped from the orientation shown in FIG. 7F. In FIG. 7G, the diaphragm 40 is shown with the first or large diaphragm opening 45 at the top of the figure, and the smaller second diaphragm opening 48 disposed at the bottom of the figure.

FIG. 7H illustrates a perspective cross-sectional or cut-away view of a portion of a diaphragm 40 comprising at least one bellows, collapsible features, accordioning layers, steps, or jogs 52 that allow the first end 42 of the diaphragm 40 to have an outer perimeter or lip 44 to fit over the first end 22 of the chamber housing or cone 20, and the second end 46 forming the second opening 46 through which the nose of the animal may be disposed, so as to enter the chamber hosing or cone 20 and receive the fluid or gas distributed to, or dispersed to within, the chamber housing 20.

FIG. 7I illustrates a side or profile cross-sectional or cut-away view of the portion of the diaphragm 40 shown in FIG. 7H.

FIG. 7J illustrates a perspective view of the anesthesia induction cone assembly 10, in which the diaphragm 40 is shown on the left side of the figure and the second end 26 of the chamber housing 20 is shown on the right side of the figure. FIG. 7J differs from FIGS. 7A and 7B in that the retaining strap 60, while coupled to the diaphragm 40, is rotated away from the chamber housing 20, so as to be positioned for being disposed over a head or neck of an animal wearing the cone assembly 10 so as to hold the cone assembly 10 in position over the animal's nose. The transition of the strap 60 from a storage position to an active or worn position may be facilitated by strap attachment device 67 positioned at or near both the first end 62 and the second end 64 of end of the strap 60, which as shown in FIG. 7J, which may be coupled with the diaphragm 40.

It will be understood that implementations of the veterinary anesthesia induction cone assembly include but are not limited to the specific components disclosed herein, as virtually any components consistent with the intended operation of various powered wall plate attachments may be utilized. Accordingly, for example, it should be understood that, while the drawings and accompanying text show and describe particular implementations, any such implementation may comprise any shape, size, style, type, model, version, class, grade, measurement, concentration, material, weight, quantity, and/or the like consistent with the intended operation of veterinary anesthesia induction cone assembly.

Furthermore, veterinary anesthesia induction cone assemblies may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular powered wall plate attachment implementations, it should be readily apparent that a number of modifications may be made without departing from the spirit thereof and that these implementations may be applied to other implementations disclosed or undisclosed. The presently disclosed powered wall plate attachments are, therefore, to be considered in all respects as illustrative and not restrictive.

What is claimed is:

1. A veterinary anesthesia induction cone assembly, comprising:
a chamber housing comprising a rim at a first end for receiving a snout of an animal and a second end opposite the first end;
a diaphragm comprising:
a compliant lip configured to be releasably and mateably coupled with the rim of the first end of the chamber housing to form a first diaphragm opening,
a second end opposite the lip and disposed within the chamber housing, the second end comprising a second diaphragm opening opposite the first diaphragm opening and configured to allow a nose of the animal to receive gas flow from the chamber housing, wherein the second diaphragm opening is smaller than the first diaphragm opening, and
a diaphragm sidewall connecting, and extending between, the first diaphragm opening and the second diaphragm opening, and
a retaining strap moveably coupled to the chamber housing and positionable between a first position for fitting the retaining strap around a head of an animal wearing the veterinary anesthesia induction cone and a second position for storing the retaining strap around the chamber housing.

2. The veterinary anesthesia induction cone assembly of claim 1, further comprising ends of the retaining strap attached to, and directly contacting, the chamber housing.

3. The veterinary anesthesia induction cone assembly of claim 1, wherein the strap further comprises a center pad disposed between the first end of the strap and the second end of the strap and a storage attachment device adjacent the center pad.

4. The veterinary anesthesia induction cone assembly of claim 1, wherein the diaphragm comprises a telescoping diaphragm with a length less than a length of the chamber housing.

5. The veterinary anesthesia induction cone assembly of claim 1, wherein the chamber housing comprises a tapered form that facilitates the stacking or nesting of multiple chamber housings with each other for compact storage, shipping, or transport.

6. The veterinary anesthesia induction cone assembly of claim 1, wherein the chamber housing comprises a frustoconical shape.

7. The veterinary anesthesia induction cone assembly of claim 1, wherein the second diaphragm opening is moveable between a first position adjacent the first diaphragm opening and a second expanded position away from the first diaphragm opening.

8. A veterinary anesthesia induction cone assembly, comprising:
a chamber housing comprising a first end for receiving a snout of an animal and a second end opposite the first end; and
a diaphragm comprising:
a first end configured to be releasably and mateably coupled with the first end of the chamber housing to form a first diaphragm opening,
a second end opposite the first end and disposed within the chamber housing, the second end comprising a second diaphragm opening opposite the first diaphragm opening and configured to allow a nose of the animal to receive gas flow from the chamber housing, and
a diaphragm sidewall connecting, and extending between, the first diaphragm opening and the second diaphragm opening.

9. The veterinary anesthesia induction cone assembly of claim 8, further comprising a retaining strap moveably coupled to the chamber housing and positionable between a first position for fitting the retaining strap around a head of an animal wearing the veterinary anesthesia induction cone and a second position for storing the retaining strap around the chamber housing.

10. The veterinary anesthesia induction cone assembly of claim 9, further comprising:
the chamber housing comprising a groove formed around a circumference of the chamber housing; and
the retaining strap comprising a ring of material mateably coupled with the groove formed around the circumference of the chamber housing.

11. The veterinary anesthesia induction cone assembly of claim 8, further comprising a 1-way port or valve disposed within the chamber housing and configured to allow for exhalation by the animal to outside of the chamber housing.

12. The veterinary anesthesia induction cone assembly of claim 8, wherein the chamber housing comprises a tapered form that facilitates the stacking or nesting of multiple chamber housings with each other for compact storage, shipping, or transport.

13. The veterinary anesthesia induction cone assembly of claim 8, wherein the second diaphragm opening is moveable between a first position adjacent the first diaphragm opening and a second expanded position away from the first diaphragm opening.

14. A veterinary anesthesia induction cone assembly, comprising:
a chamber housing comprising a first end for receiving a snout of an animal and a second end opposite the first end;
a diaphragm coupled to, and disposed within, the chamber housing, wherein the diaphragm comprises a diaphragm opening disposed within the chamber housing and configured to allow a nose of the animal to receive gas flow from the chamber housing; and
a retaining strap moveably coupled to the chamber housing and positionable between a first position for fitting the retaining strap around a head of an animal wearing the veterinary anesthesia induction cone and a second position for storing the retaining strap around the chamber housing.

15. The veterinary anesthesia induction cone assembly of claim 14, further comprising a diaphragm sidewall extending from the second diaphragm opening and configured to envelope the snout of the animal within the chamber housing.

16. The veterinary anesthesia induction cone assembly of claim 14, further comprising:
the chamber housing comprising a groove formed around a circumference of the chamber housing; and
the retaining strap comprising a ring of material mateably coupled with the groove formed around the circumference of the chamber housing.

17. The veterinary anesthesia induction cone assembly of claim 14, further comprising ends of the retaining strap attached to, and directly contacting, the chamber housing.

18. The veterinary anesthesia induction cone assembly of claim 14, wherein the strap further comprises a storage attachment device disposed between the first end of the strap and the second end of the strap.

19. The veterinary anesthesia induction cone assembly of claim 14, wherein the chamber housing comprises a tapered form that facilitates the stacking or nesting of multiple chamber housings with each other for compact storage, shipping, or transport.

20. The veterinary anesthesia induction cone assembly of claim 14, wherein the diaphragm opening is moveable between a first position adjacent the first end of the chamber housing and a second expanded position away from the first end of the chamber housing.
